(19) United States
(12) Patent Application Publication
(10) Pub. No.: US 2015/0224345 A1
(43) Pub. Date: Aug. 13, 2015

(54) ACOUSTIC SHOCK WAVE THERAPEUTIC METHODS

(71) Applicant: John Warlick, Woodstock, GA (US)

(72) Inventor: John Warlick, Woodstock, GA (US)

(21) Appl. No.: 14/180,007

(22) Filed: Feb. 13, 2014

(51) Int. Cl. A61N 7/00

(52) U.S. Cl. CPC A61N 7/00; A61N 2007/0004

(57) ABSTRACT

A method of treating a patient having a nerve injury or spinal cord injury or spinal cord lesions, or disease or any pathology that has compromised an autonomic nervous system of a patient to stimulate regeneration or reactivation of the autonomic nervous system comprises the steps of: treating the patient with injured or damaged nerves; activating an acoustic shock wave generator or source to emit acoustic shock waves from a shock wave head; and administering an effective exposure of acoustic shock waves in a pulse or wave pattern having a low energy density less than 1.0 mJ/mm² per shock wave directly onto a treatment zone in a region extending from the medulla oblongata in the lower brain stem to the lower end of the spinal cord.

### TECHNICAL FIELD

The present invention relates to improved methods and treatments to stimulate a patient's autonomic nervous system. The present invention is directed to using acoustic shock waves to stimulate an autonomic nervous system response from a patient with spinal cord nerve damage or spinal nerve lesions resulting in a degraded nervous system. In some cases, these patients are paralyzed, partially or fully.

### BACKGROUND OF THE INVENTION

The present invention employs newly discovered information and refinements in the use of acoustic shock waves to treat patients to enhance repair of damaged nerve tissue and to reactivate a degraded autonomic nervous system. The inventor has previously been awarded a number of patents using low energy acoustic shock waves to stimulate cellular responses for a variety of useful therapeutic methods.

In U.S. Pat. No. 7,470,240 B2, entitled “Pressure Pulse/Shock Wave Therapy Methods And An Apparatus For Conducting The Therapeutic Methods”, is disclosed a novel use of unfocused shock waves in a low energy range to stimulate a cellular substance. From this patent a family of treatment patents evolved. The list includes U.S. Pat. No. 7,841,995; U.S. Pat. No. 7,883,482; U.S. Pat. No. 7,905,845 all divisional applications; and U.S. Pat. No. 7,507,213 entitled “Pressure Pulse/Shock Wave Therapy Methods For Organs”; U.S. Pat. No. 7,544,171 B2 entitled “Methods for Promoting Nerve Regeneration and Neuronal Growth and Elongation”; all teaching a new useful way to deliver acoustic shock waves to achieve a healing response. Each of these patents are incorporated herein by reference in their entirety.

In addition, the present invention has recently received patents U.S. Pat. Nos. 8,257,282 and 8,535,249 for the device to perform these methods by delivering low energy unfocused acoustic shock waves to the cellular tissue being treated.

While this large volume of research has been rewarded by the granting of numerous patents, much new work has been evolving as the understanding of the technology is being applied. It is in this latest work that some, heretofore, unknown improvements and refinement have been discovered that were hidden from and unappreciated by scientists in this field.

It is an object of the present invention to disclose these new discoveries which are believed significant findings improving how the acoustic shock waves can be delivered to enhance cellular stimulation, but surprisingly how the autonomic nervous system can be manipulated using these techniques and how these new techniques can cause reparative activation of various organs and limbs of patients suffering from spinal cord injuries or disease. It is a further objective to show new regenerative actions to cause musculoskeletal regeneration to enhance pulmonary functions of these patients weakened by spinal cord injuries. These and other benefits are disclosed as summarized below.

### SUMMARY OF THE INVENTION

A method of treating a patient having a nerve injury or spinal cord injury or spinal cord lesions, or disease or any pathology that has compromised an autonomic nervous system of a patient to stimulate regeneration or reactivation of the autonomic nervous system comprises the steps of: treating the patient with injured or damaged nerves; activating an acoustic shock wave generator or source to emit acoustic shock waves from a shock wave head; and administering an effective exposure of acoustic shock waves in a pulse or wave pattern having a low energy density less than 1.0 mJ/mm² per shock wave directly onto a treatment zone in a region extending from the medulla oblongata in the lower brain stem to the lower end of the spinal cord. The administered shock waves activate an autonomic nervous system response in either a parasympathetic nervous system or a sympathetic nervous system or both systems of the autonomic nervous system. The autonomic nervous system response includes an improvement in one or more visceral functions. The improvement in one or more visceral functions includes one or more of cardio-vascular performance, digestion, respiratory performance, salivation, perspiration, pupillary dilation, micturition and sexual arousal.

The administered acoustic shock waves cause an increase of nitric oxide in the treatment zone. The administered acoustic shock waves cause a chemical release of neurotransmitters from the nerves, wherein the neurotransmitters released are epinephrine or acetylcholine or nitric oxide. The patient being treated has a spinal injury resulting in complete or partial paralysis or a patient with a compromised autonomic nervous system from any other pathology. The patient has a reduced pulmonary function. The improvement is evidenced by activation of the diaphragm muscles. The improvement is further evidenced by increased or near normal lung function and improved breathing. Additional response may result in improvement which is evidenced by active perspiration, by limb movement or by normal bladder control and urination.

### DEFINITIONS

A “curved emitter” is an emitter having a curved reflecting (or focusing) or emitting surface and includes, but is not limited to, emitters having ellipsoidal, parabolic, quasi parabolic (general paraboloid) or spherical reflector/reflecting or emitting elements. Curved emitters having a curved reflecting or focusing element generally produce waves having focused wave fronts, while curved emitters having a curved emitting surfaces generally produce wave having divergent wave fronts. Note. The shockwave does not have to be generated in a lens. A spherical wave can be generated by two opposing tips, in fluid, without a lens.

“Divergent waves” in the context of the present invention are all waves which are not focused and are not plane or nearly plane. Divergent waves also include waves which only seem to have a focus or source from which the waves are transmitted. The wave fronts of divergent waves have divergent characteristics. Divergent waves can be created in many different ways, for example: A focused wave will become divergent once it has passed through the focal point. Spherical waves are also included in this definition of divergent waves and have wave fronts with divergent characteristics.

“Extracorporeal” occurring or based outside the living body.

A “generalized paraboloid” according to the present invention is also a three-dimensional bowl. In two dimensions (in Cartesian coordinates, x and y) the formula yn=2px [with n being ≠2, but being greater than about 1.2 and smaller than 2, or greater than 2 but smaller than about 2.8]. In a generalized paraboloid, the characteristics of the wave fronts created by electrodes located within the generalized paraboloid may be corrected by the selection of (p (−z,+z)), with z being a measure for the burn down of an electrode, and n, so that phenomena including, but not limited to, burn down of the tip of an electrode (−z,+z) and/or disturbances caused by diffraction at the aperture of the paraboloid are compensated for.

A “paraboloid” according to the present invention is a three-dimensional reflecting bowl. In two dimensions (in Cartesian coordinates, x and y) the formula y2=2px, wherein p/2 is the distance of the focal point of the paraboloid from its apex, defines the paraboloid. Rotation of the two-dimensional figure defined by this formula around its longitudinal axis generates a de facto paraboloid.

“Plane waves” are sometimes also called flat or even waves. Their wave fronts have plane characteristics (also called even or parallel characteristics). The amplitude in a wave front is constant and the “curvature” is flat (that is why these waves are sometimes called flat waves). Plane waves do not have a focus to which their fronts move (focused) or from which the fronts are emitted (divergent). “Nearly plane waves” also do not have a focus to which their fronts move (focused) or from which the fronts are emitted (divergent). The amplitude of their wave fronts (having “nearly plane” characteristics) is approximating the constancy of plain waves. “Nearly plane” waves can be emitted by generators having pressure pulse/shock wave generating elements with flat emitters or curved emitters. Curved emitters may comprise a generalized paraboloid that allows waves having nearly plane characteristics to be emitted.

A “pressure pulse” according to the present invention is an acoustic pulse which includes several cycles of positive and negative pressure. The amplitude of the positive part of such a cycle should be above about 0.1 MPa and its time duration is from below a microsecond to about a second. Rise times of the positive part of the first pressure cycle may be in the range of nano-seconds (ns) up to some milli-seconds (ms). Very fast pressure pulses are called shock waves. Shock waves used in medical applications do have amplitudes above 0.1 MPa and rise times of the amplitude are below 100 ns. The duration of a shock wave is typically below 1-3 micro-seconds (μs) for the positive part of a cycle and typically above some micro-seconds for the negative part of a cycle.

Waves/wave fronts described as being “focused” or “having focusing characteristics” means in the context of the present invention that the respective waves or wave fronts are traveling and increase their amplitude in direction of the focal point. Per definition the energy of the wave will be at a maximum in the focal point or, if there is a focal shift in this point, the energy is at a maximum near the geometrical focal point. Both the maximum energy and the maximal pressure amplitude may be used to define the focal point.

### BRIEF DESCRIPTION OF THE DRAWINGS

The invention will be described by way of example and with reference to the accompanying drawings in which:

FIG. 1a is a simplified depiction of a pressure pulse/shock wave (PP/SW) generator with focusing wave characteristics.

FIG. 1b is a simplified depiction of a pressure pulse/shock wave generator with plane wave characteristics.

FIG. 1c is a simplified depiction of a pressure pulse/shock wave generator with divergent wave characteristics.

FIG. 2a is a simplified depiction of a pressure pulse/shock wave generator having an adjustable exit window along the pressure wave path. The exit window is shown in a focusing position.

FIG. 2b is a simplified depiction of a pressure pulse/shock wave generator having an exit window along the pressure wave path. The exit window as shown is positioned at the highest energy divergent position.

FIG. 2c is a simplified depiction of a pressure pulse/shock wave generator having an exit window along the pressure wave path. The exit window is shown at a low energy divergent position.

FIG. 3 is a simplified depiction of an electro-hydraulic pressure pulse/shock wave generator having no reflector or focusing element. Thus, the waves of the generator did not pass through a focusing element prior to exiting it.

FIG. 4a is a simplified depiction of a pressure pulse/shock wave generator having a focusing element in the form of an ellipsoid. The waves generated are focused.

FIG. 4b is a simplified depiction of a pressure pulse/shock wave generator having a parabolic reflector element and generating waves that are disturbed plane.

FIG. 4c is a simplified depiction of a pressure pulse/shock wave generator having a quasi parabolic reflector element (generalized paraboloid) and generating waves that are nearly plane/have nearly plane characteristics.

FIG. 4d is a simplified depiction of a generalized paraboloid with better focusing characteristic than a paraboloid in which n=2. The electrode usage is shown. The generalized paraboloid, which is an interpolation (optimization) between two optimized paraboloids for a new electrode and for a used (burned down) electrode is also shown.

FIG. 5 is a simplified depiction of a pressure pulse/shock wave generator being connected to a control/power supply unit.

FIG. 6 is a simplified depiction of a pressure pulse/shock wave generator comprising a flat EMSE (electromagnetic shock wave emitter) coil system to generate nearly plane waves as well as an acoustic lens. Convergent wave fronts are leaving the housing via an exit window.

FIG. 7 is a simplified depiction of a pressure pulse/shock wave generator having a flat EMSE coil system to generate nearly plane waves. The generator has no reflecting or focusing element. As a result, the pressure pulse/shock waves are leaving the housing via the exit window unfocused having nearly plane wave characteristics.

FIG. 8 is a simplified depiction of a pressure pulse/shock wave generator having a flat piezoceramic plate equipped with a single or numerous individual piezoceramic elements to generate plane waves without a reflecting or focusing element. As a result, the pressure pulse/shock waves are leaving the housing via the exit window unfocused having nearly plane wave characteristics.

FIG. 9 is a simplified depiction of a pressure pulse/shock wave generator having a cylindrical EMSE system and a triangular shaped reflecting element to generate plane waves. As a result, the pressure pulse/shock waves are leaving the housing via the exit window unfocused having nearly plane wave characteristics.

FIG. 10 is a simplified depiction of a pressure pulse/shock wave (PP/SW) generator with focusing wave characteristics shown focused with the focal point or geometrical focal volume being on an organ, the focus being targeted on the location X₀.

FIG. 11 is a simplified depiction of a pressure pulse/shock wave (PP/SW) generator with the focusing wave characteristics shown wherein the focus is located a distance X, from the location X₀ of an organ wherein the converging waves impinge the organ.

FIG. 12 is a simplified depiction of a pressure pulse/shock wave (PP/SW) generator with focusing wave characteristics shown wherein the focus is located a distance X₂ from the mass location X₀ wherein the emitted divergent waves impinge the organ.

FIG. 13 shows a patient being treated extracorporeally with shock waves being transmitted through the skin and spinal bone tissue to the neurological region to be treated.

FIG. 14 shows a diagram from Gray's Anatomy showing the various organs, glands and tissues that are connected by the nerves and controlled by the regions of the medulla oblongata to the end of the spinal cord.

### DETAILED DESCRIPTION OF THE INVENTION

The present invention is directed to using acoustic shock waves to stimulate an autonomic nervous system response from a patient with spinal cord nerve damage or spinal nerve lesions resulting in a degraded nervous system. In some cases, these patients are paralyzed, partially or fully.

Autonomic nervous system (ANS) is the part of the peripheral nervous system that acts as a control system, functioning largely below the level of consciousness, and controls visceral functions. The ANS affects heart rate, digestion, respiratory rate, salivation, perspiration, pupillary dilation, micturition (urination), and sexual arousal. Most autonomous functions are involuntary but they can often work in conjunction with the somatic nervous system which gives voluntary control. Everyday examples include breathing, swallowing, and sexual arousal, and in some cases functions such as heart rate.

Within the brain, the ANS is located in the medulla oblongata in the lower brainstem. The medulla's major ANS functions include respiration, cardiac regulation, vasomotor activity and certain reflex actions such as coughing, sneezing, vomiting and swallowing. These then subdivide into other areas and are also linked to ANS subsystems and nervous systems external to the brain.

The ANS is classically divided into two subsystems: the parasympathetic nervous system (PSNS) and sympathetic nervous system (SNS), which operate independently in some functions and interact co-operatively in others. In many cases, the two have “opposite” actions where one activates a physiological response and the other inhibits it. The enteric nervous system is also sometimes considered part of the autonomic nervous system, and sometimes considered an independent system.

In general, ANS functions can be divided into sensory (afferent) and motor (efferent) subsystems. Within both, there are inhibitory and excitatory synapses between neurons. Relatively recently, a third subsystem of neurons that have been named ‘non-adrenergic and non-cholinergic’ neurons. The neurons use nitric oxide as a neurotransmitter and have been described and found to be integral in autonomic function, in particular in the gut and the lungs.

The sympathetic division has thoracolumbar “outflow”, meaning that the neurons begin at the thoracic and lumbar (T1-L2/3) portions of the spinal cord. The parasympathetic division has craniosacral “outflow”, meaning that the neurons begin at the cranial nerves (Cranial nerves III, VII, IX, and X) and sacral (S2-S4) spinal cord.

The ANS is unique in that it requires a sequential two-neuron efferent pathway; the preganglionic neuron must first synapse onto a postganglionic neuron before innervating the target organ. The preganglionic, or first, neuron will begin at the “outflow” and will synapse at the postganglionic, or second, neuron's cell body. The postganglionic neuron will then synapse at the target organ.

The sympathetic division (thoracolumbar outflow) consists of cell bodies in the lateral horn of the spinal cord (intermediolateral cell columns) from T1 to L2/3.

The parasympathetic division (craniosacral outflow) consists of cell bodies from one of two locations: the brainstem (Cranial Nerves III, VII, IX, X) or the sacral spinal cord (S2, S3, S4). These are the preganglionic neurons, which synapse with postganglionic neurons in these locations. Visceral sensory information constantly and unconsciously modulates the activity of the motor neurons of the ANS.

Motor neurons of the ANS are also located in ganglia of the PNS, called “autonomic ganglia.” They belong to three categories with different effects on their target organs.

Sympathetic ganglia are located in two sympathetic chains close to the spinal cord: the prevertebral and pre-aortic chains. Parasympathetic ganglia, in contrast, are located in close proximity to the target organ: the submandibular ganglion close to salivary glands, paracardiac ganglia close to the heart, etc. Enteric ganglia, which as their name implies innervate the digestive tube, are located inside its walls and collectively contain as many neurons as the entire spinal cord, including local sensory neurons, motor neurons and interneurons. It is the only truly autonomous part of the ANS and the digestive tube can function surprisingly well even in isolation. For that reason the enteric nervous system has been called “the second brain.”

The activity of autonomic ganglionic neurons is modulated by “preganglionic neurons” located in the central nervous system (CNS). Preganglionic sympathetic neurons are in the spinal cord, at thoraco-lumbar levels. Preganglionic parasympathetic neurons are in the medulla oblongata, forming visceral motor nuclei: the dorsal motor nucleus of the vagus nerve (dmnX), the nucleus ambiguus, and the salivatory nuclei, and in the sacral spinal cord. Enteric neurons are also modulated by input from the CNS and from preganglionic neurons located, like parasympathetic ones, in the medulla oblongata (in the dmnX).

Sympathetic and parasympathetic divisions typically function in opposition to each other. But this opposition is better termed complementary in nature rather than antagonistic. For an analogy, one may think of the sympathetic division as the accelerator and the parasympathetic division as the brake. The sympathetic division typically functions in actions requiring quick responses. The parasympathetic division functions with actions that do not require immediate reaction. In general, these two systems should be seen as permanently modulating vital functions, in usually antagonistic fashion, to achieve homeostasis.

Sympathetic nervous system promotes a quick response, corresponds with arousal and energy generation, and inhibits digestion. Diverts blood flow away from the gastro-intestinal (GI) tract and skin via vasoconstriction. Blood flow to skeletal muscles and the lungs is enhanced by as much as 1200% in the case of skeletal muscles. Dilates bronchioles of the lung, which allows for greater alveolar oxygen exchange. Increases heart rate and the contractility of cardiac cells (myocytes), thereby providing a mechanism for enhanced blood flow to skeletal muscles. Dilates pupils and relaxes the ciliary muscle to the lens, allowing more light to enter the eye and far vision. Provides vasodilation for the coronary vessels of the heart. Constricts all the intestinal sphincters and the urinary sphincter. Inhibits peristalsis. Stimulates orgasm.

Parasympathetic nervous system promotes a non-immediate response, such as calming of the nerves return to regular function, and enhances digestion. The parasympathetic nerves dilate blood vessels leading to the GI tract, increasing blood flow. This is important following the consumption of food, due to the greater metabolic demands placed on the body by the gut. The parasympathetic nervous system can also constrict the bronchiolar diameter when the need for oxygen has diminished. Dedicated cardiac branches of the vagus and thoracic spinal accessory nerves impart parasympathetic control of the heart (myocardium). During accommodation, the parasympathetic nervous system causes constriction of the pupil and contraction of the ciliary muscle to the lens, allowing for closer vision. The parasympathetic nervous system stimulates salivary gland secretion, and accelerates peristalsis, mediating digestion of food and, indirectly, the absorption of nutrients. It is also involved in the erection of genital tissues via the pelvic splanchnic nerves 2-4 and is responsible for stimulating sexual arousal.

At the effector organs, sympathetic ganglionic neurons release noradrenaline (norepinephrine), along with other cotransmitters such as ATP, to act on adrenergic receptors, with the exception of the sweat glands and the adrenal medulla:

Acetylcholine is the preganglionic neurotransmitter for both divisions of the ANS, as well as the postganglionic neurotransmitter of parasympathetic neurons. Nerves that release acetylcholine are said to be cholinergic. In the parasympathetic system, ganglionic neurons use acetylcholine as a neurotransmitter to stimulate muscarinic receptors.

At the adrenal medulla, there is no postsynaptic neuron. Instead the presynaptic neuron releases acetylcholine to act on nicotinic receptors. Stimulation of the adrenal medulla releases adrenaline (epinephrine) into the bloodstream, which acts on adrenoceptors, producing a widespread increase in sympathetic activity.

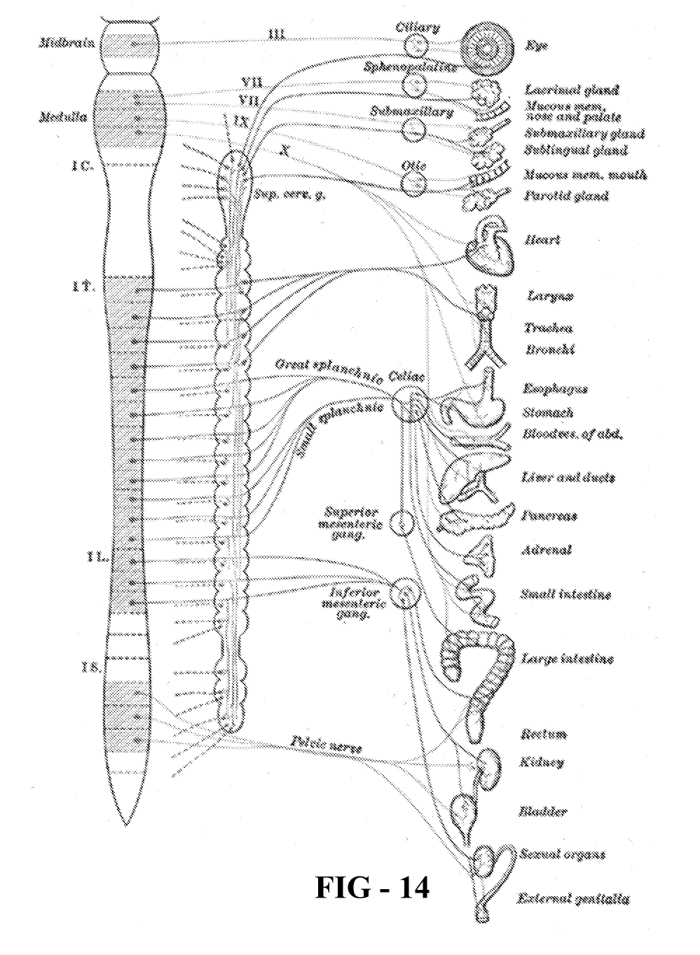

The viscera are mainly innervated parasympathetically by the vagus nerve and sympathetically by the splanchnic nerves. The sensory part of the latter reaches the spinal column at certain spinal segments. Pain in any viscera is perceived as referred pain, more specifically pain from the dermatome corresponding to the spinal segment. To better appreciate the overall connections between the lower brain stem and the nerves of the spinal cord, a diagram from Gray's Anatomy is provided as FIG. 14. This chart shows the various organs, glands and tissues that are connected by the nerves and controlled by the regions of the medulla oblongata to the end of the spinal cord.

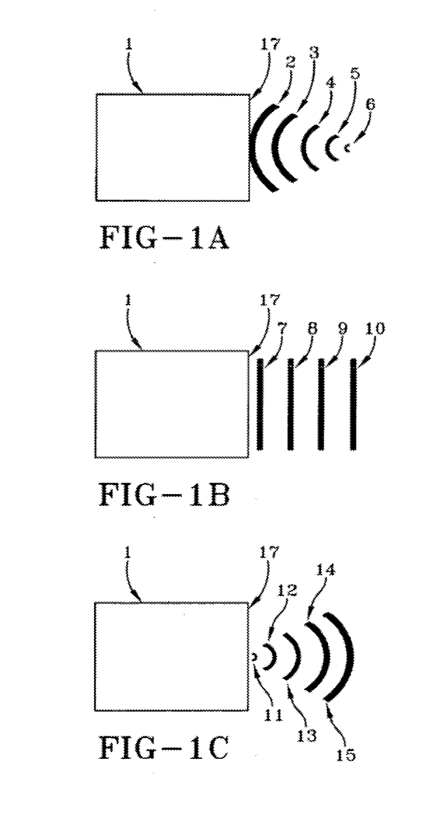

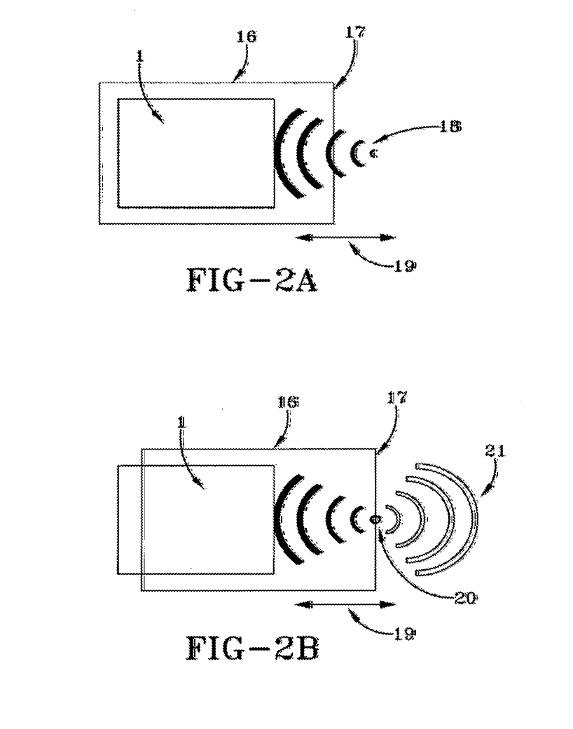

The present invention relates to the use of various therapeutic pressure pulse wave patterns or acoustic shock wave patterns as illustrated in FIGS. 1-12 for treating patients having nerve damage or nerve lesions in the spinal cord region that have degraded the performance of the autonomic nervous system. Each illustrated wave pattern will be discussed later in the description; however, the use of each has particularly interesting beneficial features that are a remarkably valuable new tool in the fight against such diseases.

With reference to FIG. 13, a perspective view of a portion of the treatment region 200 is shown between the lower brain stem to the lower end of the spinal cord. The neurological tissue 100, more commonly referred to as the medulla oblongata and the spinal cord tissue, is the principal source of neurological connectivity activity between the organs and the limbs. With further reference to FIG. 13, the patient P who has nerve damage or nerve lesions due to a spinal cord injury, disease, or any other pathology, is positioned on a table T preferably face down lying on the stomach. A shock wave applicator head 43 is brought into contact with the skin Pₛ preferably an acoustic gel is used to enhance the transmission of the shock waves 200 through the body down to the subsurface nerve tissue 100 in the region 101 at the lower brain stem to the end of the spinal cord. The shock wave applicator head 43 is connected via cabling 42 to a power generating unit 41 as shown. The shock wave applicator head 43 can be attached rigidly to a fixture or stand 44 as illustrated or alternatively can be hand held and manipulated across the skin Pₛ to drive the shock waves 200 in the direction the shock wave head 43 is pointed to activate a response to the autonomic nervous system (ANS).

Shock waves are a completely different technology and a quantum leap beyond other forms of neurological treatments. The mechanism of shock waves is far from being understood, but is known to cause new blood vessels to grow in an area of treatment and regenerate bony tissue. In the present invention shock waves are used to treat patients with nerve damage or neurological disease by not only regenerating or repairing the neurological tissue or creating new nerve architecture, but most remarkably reactivating a degraded autonomic nervous system response. This is a phenomenal advancement in the current approach which generally avoids difficult surgery or can be used in conjunction with a surgically repaired injury as a complimentary treatment to such surgery. If surgery could be replaced in many cases, it would save millions of dollars, gain wide acceptance (non-invasive) and be a tremendous benefit to patients worldwide.

The present invention employs the use of pressure pulses or shock waves to stimulate a neuron or cellular nerve response stimulating the autonomic system to respond starting a tissue regenerative healing process that activates the tissue or nerve cells not only of damaged nerves, but also initiates a systemic healing process to re-energize affected organs and muscle tissue through an improvement in the degraded autonomic nervous system. With spinal cord injuries, a patient without the ability to move can quickly experience muscle atrophy. This causes many systems in the body to be adversely affected, not the least of which is the ability of the diaphragm muscles to assist the patient's breathing and lung capacity which affects the ability to generate oxygen in the lungs to oxygenate the blood vessels. As a result, the patient's entire physical system can degrade rapidly.

In the pressure pulse or shock wave method of treating a tissue, an organ or the entire body of a human patient with a risk of degenerative neurological or nerve damage or post-occurrence of such damage requires the patient to be positioned in a convenient orientation to permit the source of the emitted waves to most directly send the waves to the target site to initiate pressure pulse or shock wave stimulation of the target area or zone with minimal, preferably with little or no obstructing features in the path of the emitting source or lens. Assuming the treatment region is accessible through an open surgical access region then the shock wave head 43 can be inserted and placed directly on or adjacent to the treatment region 200. Alternatively the shock wave head 43 can be placed externally on the back and transmit the emitted shock wave patterns through the skin, spinal bone tissue 116 for example and into the adjacent nerve tissue 100 to be treated, as shown in FIG. 13. In the case of extracorporeal non-invasive treatments of damaged nerves, preferably the outer skin tissue is pressed against the treatment region to insure the transmission loss is minimal. In some cases the treatment zone may benefit or require numbing prior to treatments in advance of surgical procedures. This is particularly true after a number of treatments over a period of time, because as the nerves heal, the patient's sensation of pain will be reacquired. This is particularly true if the use of high energy focused waves are being transmitted through the spinal bone tissue to stimulate the sensitive nerves in the treatment area. Assuming the target area or site is within a projected area of the wave transmission, a single transmission dosage of wave energy may be used. The transmission dosage can be from a few seconds to 20 minutes or more dependent on the condition. Preferably the waves are generated from an unfocused or focused source. The unfocused waves can be divergent, planar or near planar and having a low pressure amplitude and density in the range of 0.00001 mJ/mm2 to 1.0 mJ/mm2 or less, most typically below 0.2 mJ/mm2. The focused source preferably can use a diffusing lens or have a far-sight focus to minimize if not eliminate having the localized focus point within the tissue. Preferably the focused shock waves are used at a similarly effective low energy transmission or alternatively can be at higher energy but wherein the tissue target site is disposed pre-convergence inward of the geometric focal point of the emitted wave transmission. In treating some hard to penetrate regions, the pressure pulse more preferably is a high energy target focused wave pattern which can effectively penetrate through outer structures prior to being dampened while still exposing the nerves or neurons to activating pressure pulses or shock waves. This emitted energy preferably stimulates the cells with minimal rupturing of cellular membranes. The surrounding healthy cells in the region treated are activated initiating a defense mechanism response to assist in eradication of the unwanted infection or diseased tissue while stimulating new growth and enhanced autonomic nervous system performance.

These shock wave energy transmissions are effective in stimulating a cellular response and can be accomplished without creating excessive cavitation bubbles in the tissue of the target site when employed in other than site targeted high energy focused transmissions. This effectively insures the spinal cord tissue or lower brain tissue does not have to experience the sensation of excessive hemorrhaging so common in the use of higher energy focused wave forms having a focal point at or within the targeted treatment site.

If the target site is the lower brain stem or the spinal cord subjected to a surgical procedure exposing at least some if not all of the tissue, then the target site may be such that the patient or the generating source must be reoriented relative to the site and a second, third or more treatment dosages can be administered. The fact that some if not all of the dosage can be at a low energy the common problem of localized hemorrhaging can be reduced making it more practical to administer multiple dosages of waves from various orientations to further optimize the treatment and cellular stimulation of the target site. Heretofore focused high energy multiple treatments induced pain and discomfort to the patient. The use of low energy focused or un-focused waves at the target site enables multiple sequential treatments with minimal pain. While pain is a sensation some paralyzed patients may not experience during early treatments, it is still preferable to keep the energy density at or below 1.0 mJ/mm².

The present method may need precise site location and can be used in combination with such known devices as ultrasound, cat-scan or x-ray imaging if needed. The physician's general understanding of the anatomy of the patient may be sufficient to locate the target area to be treated. This is particularly true when the exposed nerve tissue or portion of the trauma to the body or organ is visually within the surgeon's line of sight and this permits the lens or cover of the emitting shock wave source to impinge on the affected organ or tissue directly or through a transmission enhancing gel, water or fluid medium during the pressure pulse or shock wave treatment. The treated area can withstand a far greater number of shock waves based on the selected energy level being emitted. For example at very low energy levels the stimulation exposure can be provided over prolonged periods as much as 20 minutes if so desired. At higher energy levels the treatment duration can be shortened to less than a minute, less than a second if so desired. The limiting factor in the selected treatment dosage is minimization of surrounding cell hemorrhaging and other kinds of damage to the surrounding cells or tissue while still providing a stimulating stem cell activation or a cellular release or activation of proteins such as brain derived neurotropic factor (BDNF) or VEGF and other growth factors while simultaneously germicidally attacking the degenerative tissue or infectious bacteria at the wound site.

Due to the wide range of beneficial treatments available it is believed preferable that the optimal use of one or more wave generators or sources should be selected on the basis of the specific application. Wherein relatively small target sites may involve a single wave generator placed on an adjustable manipulator arm. A key advantage of the present inventive methodology is that it is complimentary to conventional medical procedures. In the case of any operative surgical procedure the surgical area of the patient can be bombarded with these energy waves to stimulate cellular release of healing agents and growth factors. This will dramatically reduce the healing process time. Most preferably such patients may be provided more than one such treatment with an intervening dwell time for cellular relaxation prior to secondary and tertiary post-operative treatments.

The underlying principle of these pressure pulse or shock wave therapy methods is to enrich the treatment area directly and to stimulate the body's own natural healing capability by causing the degraded autonomic nervous system to activate a response. This is accomplished by deploying shock waves to stimulate strong cells in the surrounding tissue to activate a variety of responses. The acoustic shock waves transmit or trigger what appears to be a cellular communication throughout the entire anatomical structure, this activates a generalized cellular response at the treatment site, in particular, but more interestingly a systemic response in areas more removed from the wave form pattern. This is believed to be one of the reasons molecular stimulation can be conducted at threshold energies heretofore believed to be well below those commonly accepted as required. Accordingly not only can the energy intensity be reduced in some cases, but also the number of applied shock wave impulses can be lowered from several thousand to as few as one or more pulses and still yield a beneficial stimulating response. The key is to provide at least a sufficient amount of energy to activate healing reactions.

Nerve Lesion in Continuity: Peripheral nerve lesions with preserved continuity of the nerve trunk but loss of distal function to varying extents constitute one of the greatest challenges in peripheral nerve surgery. Such partial loss of function might result from subtotal nerve transections, blunt nerve trauma or traction injuries. Various fiber components of the nerve trunk can, in such cases, present all stages from simple neurapraxia (Sunderland grade 1) to neurotmesis (Sunderland grades 3-5). While some axons may be transected or ruptured, others may be compressed by interneural scar or compromised by vascular insufficiency. The approach to this type of injury, also called “neuroma in continuity” is extremely difficult. In these cases the surgeon may supply collagenase to the zone of injury, in accordance with the present invention. Surgical exploration, including neurolysis or resection and reconstruction, might also be indicated if the permanent situation cannot be accepted. In such cases, applying collagenase at the point of surgical intervention facilitates nerve regeneration.

The surgeon, if experienced with the type of lesion, may by inspection under high magnification be able to gauge to some extent which fascicles are healthy and should be spared and which are damaged and should be resected and replaced. However, with this method the findings can often be misleading and methods for intraoperative assessment of fiber function with electrophysiological recording techniques have been developed. Kline et al. (1968, 19691 1972) introduced techniques for intraoperative neurophysiological assessment of the extent of the lesion by stimulating and recording from whole nerves With the development of microsurgical techniques, more refined methods for stimulation and recording from individual fascicles or fascicular groups became available. Hakstian (1968) introduced a method of stimulating motor and sensory fascicles separately in the proximal and distal nerve segments to improve accuracy in experimental nerve suture, and similar techniques have long been utilized to assess the quality of nerve regeneration following various types of nerve repair (Terzis et al., 1975, 1976; Terzis & Williams, 1976).

On the basis of these investigations, single fascicular recordings have been successfully used as an intraoperative diagnostic tool in microsurgical repair of nerve lesions in continuity (Kline & Nulsen, 1972; Williams & Terzis, 1976; Kline, 1980; Terzis et al., 1980). According to these principles, single fascicles or, if that is not possible, groups of fascicles are freed by dissection and isolated proximal and distal to the lesion Each individual fascicle is lifted onto stimulating and recording electrodes, electrical stimuli are delivered proximally and a nerve compound action potential (CAP) is recorded distally to the lesion. On the basis of the conduction velocity as well as the shape and amplitude of the wave form, the degree of nerve injury can be assessed and a decision made regarding the treatment of the fascicle. If there is a measurable response, intraneural neurolysis might be justified while absence of any response might indicate resection and grafting of the damaged fascicle.

The present invention can be used in combination with each of these nerve repair techniques and exposure to such pressure pulses or shock waves greatly accelerate the nerve repair healing time which accordingly enhances the likelihood of successful recovery of nerve function.

In U.S. Pat. No. 7,544,171 B2, clinical rat studies showed remarkable re-growth of cut sciatic nerves has been demonstrated. The study involved cutting about 1.5 cm of the sciatic nerve, turning it 180° and suturing the cut ends back to the nerve (this model represents a nerve graft), closing the skin, followed by localized treatment using the acoustic shock waves. Co-inventor, Dr. Wolfgang Schaden, found that the nerves reattached/regenerated themselves better in cases where shock waves were applied. In addition, it was found that treated rats had a higher concentration of a certain protein in the brain that is common with well-trained rats (i.e. rats undergoing physiotherapy).

The trial was a 3 tailed study: 1st group of rats: dissection of the sciatic nerve and immediate microsurgical suture of the nerve. This was the control group. 2nd group: this group had the same procedure but after suturing the skin immediately shockwaves were applied. 3rd group: resection of 1.5 cm of the sciatic nerve and microsurgical suture upside-down (nerve graft model). After suturing the skin immediately shockwave therapy. Till now we have the following results: Group 1 had the expected results of sutured nerves (compared to historical study groups). Group 2 and even group 3 were clinically better than group 1. Group 2 and 3 were also better in electromyographical examinations. Both shockwave groups had significant higher levels of BDNF as the control group, but even higher levels than trained rats (based on historical comparison to trials that have been previously performed).

Dr. Robert Schmidhammer who performed the nerve trials in Austria found the protein he could prove to be produced in the brain of the rats of the shock wave therapy is called brain derived neurotropic factor (BDNF). The concentration of this protein in the shock wave treated rats was even higher than in trained rats.

These studies relied on the stimulation of the rats own natural healing ability after exposure to a shock wave treatment. The control group of rats had generally a failure to reattach and as expected no return of nerve function. This exposure to shock waves enhancing the neurological brain activity in the treated rats proved the overall systemic response of the nervous system to regenerative growth and repair after shock wave exposure at least on lower mammals such as rats.

This finding has led to the projected use of such treatments on humans for regenerative repair of degenerative conditions, the rat clinical studies indicated similar improvements could be anticipated in primates including humans.

The use of acoustic shock waves shows that growth factors are released which are indicative that otherwise dormant cells within the nerve tissue appear to be activated which leads to the remarkable ability of the targeted area to generate new growth or to regenerate weakened vascular networks or blood circulation in for example to assist in nerve regeneration. This finding leads to a complimentary use of shock wave nerve therapy in combination with stem cell therapies that effectively activate or trigger stem cells within the body to more rapidly replicate enhancing nerve repair. The ability to stimulate stem cells within the patient's own body activating the naturally occurring stem cells or stem cells that have been introduced to the patient as part of a treatment beneficially utilizing stem cells has significant clinical value.

The use of shock wave therapy requires a fundamental understanding of focused and unfocused shock waves, coupled with a more accurate biological or molecular model.

Focused shock waves are focused using ellipsoidal reflectors in electromechanical sources from a cylindrical surface or by the use of concave or convex lenses. Piezoelectric sources often use spherical surfaces to emit acoustic pressure waves which are self-focused and have also been used in spherical electromagnetic devices.

The biological model proposed by co-inventor Wolfgang Schaden provides a whole array of clinically significant uses of shock wave therapy.

Accepting the biological model as promoted by W. Schaden, the peak pressure and the energy density of the shock waves can be lowered dramatically. Activation of the body's healing mechanisms will be seen by in growth of new blood vessels and the release of growth factors.

The biological model motivated the design of sources with low pressure amplitudes and energy densities. First: spherical waves generated between two tips of an electrode; and second: nearly even waves generated by generalized parabolic reflectors. Third: divergent shock front characteristics are generated by an ellipsoid behind F2. Unfocused sources are preferably designed for extended two dimensional areas/volumes like skin. The unfocused sources can provide a divergent wave pattern a planar or a nearly planar wave pattern and can be used in isolation or in combination with focused wave patterns yielding to an improved therapeutic treatment capability that is non-invasive with few if any disadvantageous contraindications. Alternatively a focused wave emitting treatment may be used wherein the focal point extends preferably beyond the target treatment site, potentially external to the patient. This results in the reduction of or elimination of a localized intensity zone with associated noticeable pain effect while providing a wide or enlarged treatment volume at a variety of depths more closely associated with high energy focused wave treatment. The utilization of a diffuser type lens or a shifted far-sighted focal point for the ellipsoidal reflector enables the spreading of the wave energy to effectively create a convergent but off target focal point. This insures less tissue trauma while insuring cellular stimulation to enhance the healing process and control the migration or spreading of the infection within the host

The unfocused shock waves can be of a divergent wave pattern, planar or near planar pattern preferably convergent diffused or far-sighted wave pattern, of a low peak pressure amplitude and density. Typically the energy density values range as low as 0.000001 mJ/mm2 and having a high end energy density of below 1.0 mJ/mm2, preferably 0.20 mJ/mm2 or less. The peak pressure amplitude of the positive part of the cycle should be above 1.0 and its duration is below 1-3 microseconds.

The treatment depth can vary from the surface to the full depth of the treated organ. The treatment site can be defined by a much larger treatment area than the 0.10-3.0 cm2 commonly produced by focused waves. The above methodology is particularly well suited for surface as well as sub-surface soft tissue organ treatments like the brain.

The above methodology is valuable in generation of nerve tissue, vascularization and may be used in combination with stem cell therapies as well as regeneration of damaged nerve or neurological tissue and vascularization.

The methodology is useful in (re)vascularization and regeneration of not only neurological tissue such as the brain, but also the heart, liver, kidney, skin, urological organs, reproductive organs, digestive tract and muscle tissue.

The methodology is useful in stimulating enforcement of defense mechanisms in tissue cells to fight infections from bacteria and can be used germicidally to treat or cleanse wounds or other infected or degenerative target sites which is a primary concern in the case of treating human neurological diseases such as Alzheimer's disease, Parkinson's or ALS, resulting from such exposures to infectious or degenerative type agents.

While the above listed indications cited above are not exhaustive nor intended to be limiting, it is exemplary of the wide range of beneficial uses of high energy focused or low energy and amplitude unfocused divergent, planar or nearly planar shock waves, convergent shock waves, diffused shock waves or a combination of shock wave types in the treatment of humans and other mammals that are exposed to a neurological trauma or disease affecting the nervous system or are at high risk to be so exposed as the result of a high potential genetic pre-disposition to such diseases.

A most significant method of preventive medicine can be practiced that is fully enabled by the use of these relatively low amplitude and pressure shock waves. The method includes the steps of identifying high risk patients for a variety of potential risk conditions. Such condition could be by way of example, any degenerative neurological disease or loss of feeling or circulation in a target region. After identifying a risk prone candidate providing one or a series of two or more exposure treatments with focused or unfocused, divergent, planar or near planar shock waves or convergent far-sighted focused shock waves or diffused shock waves to the treatment site, in this example the region surrounding or in proximity to an occurrence risk location. Then after treatments the physician can optionally ultrasound visually or otherwise determine the increase in regeneration or vascularization in the treated tissue after a period of time. Assuming an initial baseline determination of the neurological cell or nerve tissue regeneration or vascularization had been initially conducted an estimate or calculation of dosage requirements can be made. This procedure can be used for any at risk condition. After a surgical repair procedure the surrounding tissues can be post-operatively shock wave treated as well.

The implications of using the (re)generative features of this type of shock wave therapy are any weakened organ or tissue can be strengthened to the point of reducing or eliminating the risk of irreparable damage or failure as a result of microbial infections or genetic pre-disposition.

The stimulation of growth factors and activation of healing acceleration within the cells of the treated tissues is particularly valuable to host patients and other high risk factor subjects wherein conventional treatments have been unsuccessful.

Even more striking as mentioned earlier, early prevention therapies can be employed to stimulate tissue or organ modeling to be maintained within acceptable ranges prior to an exposure to a degenerative failure. This is extremely valuable in the prevention of spreading the infection or degenerative condition for example. The methods would be to identify at risk patients with a known exposure risk, and subjecting that patient to therapeutic shock wave therapy for the purpose of stimulating neurological tissue repair or regeneration effectively remodeling the patient's susceptible organs to be within accepted functional parameters prior to irreparable degeneration. The objective being to preventively stimulate cellular tissue repairs to preemptively avoid a degenerative condition from occurring which may result in the onset of a degenerative condition which can require invasive surgical procedures.

This preventive therapy is most needed to combat conditions which left untreated results in cellular destruction or any other degenerative conditions

FIG. 1a is a simplified depiction of the a pressure pulse/shock wave (PP/SW) generator, such as a shock wave head, showing focusing characteristics of transmitted acoustic pressure pulses. Numeral 1 indicates the position of a generalized pressure pulse generator, which generates the pressure pulse and, via a focusing element, focuses it outside the housing to treat diseases. The affected tissue or organ is generally located in or near the focal point which is located in or near position 6. At position 17 a water cushion or any other kind of exit window for the acoustical energy is located.

FIG. 1b is a simplified depiction of a pressure pulse/shock wave generator, such as a shock wave head, with plane wave characteristics. Numeral 1 indicates the position of a pressure pulse generator according to the present invention, which generates a pressure pulse which is leaving the housing at the position 17, which may be a water cushion or any other kind of exit window. Somewhat even (also referred to herein as “disturbed”) wave characteristics can be generated, in case a paraboloid is used as a reflecting element, with a point source (e.g. electrode) that is located in the focal point of the paraboloid. The waves will be transmitted into the patient's body via a coupling media such as, e.g., ultrasound gel or oil and their amplitudes will be attenuated with increasing distance from the exit window 17.

FIG. 1c is a simplified depiction of a pressure pulse shock wave generator (shock wave head) with divergent wave characteristics. The divergent wave fronts may be leaving the exit window 17 at point 11 where the amplitude of the wave front is very high. This point 17 could be regarded as the source point for the pressure pulses. In FIG. 1c the pressure pulse source may be a point source, that is, the pressure pulse may be generated by an electrical discharge of an electrode under water between electrode tips. However, the pressure pulse may also be generated, for example, by an explosion, referred to as a ballistic pressure pulse. The divergent characteristics of the wave front may be a consequence of the mechanical setup shown in FIG. 2b.

FIG. 2a is a simplified depiction of a pressure pulse/shock wave generator (shock wave head) according to the present invention having an adjustable or exchangeable (collectively referred to herein as “movable”) housing around the pressure wave path. The apparatus is shown in a focusing position. FIG. 2a is similar to FIG. 1a but depicts an outer housing (16) in which the acoustical pathway (pressure wave path) is located. In a preferred embodiment, this pathway is defined by especially treated water (for example, temperature controlled, conductivity and gas content adjusted water) and is within a water cushion or within a housing having a permeable membrane, which is acoustically favorable for the transmission of the acoustical pulses. In certain embodiments, a complete outer housing (16) around the pressure pulse/shock wave generator (1) may be adjusted by moving this housing (16) in relation to, e.g., the focusing element in the generator. However, as the person skilled in the art will appreciate, this is only one of many embodiments of the present invention. While the figure shows that the exit window (17) may be adjusted by a movement of the complete housing (16) relative to the focusing element, it is clear that a similar, if not the same, effect can be achieved by only moving the exit window, or, in the case of a water cushion, by filling more water in the volume between the focusing element and the cushion. FIG. 2a shows the situation in which the arrangement transmits focused pressure pulses.

FIG. 2b is a simplified depiction of the pressure pulse/shock wave generator (shock wave head) having an adjustable or exchangeable housing around the pressure wave path with the exit window 17 being in the highest energy divergent position. The configuration shown in FIG. 2b can, for example, be generated by moving the housing (16) including the exit window (17), or only the exit window (17) of a water cushion, towards the right (as shown in the Figure) to the second focus f2 (20) of the acoustic waves. In a preferred embodiment, the energy at the exit window will be maximal. Behind the focal point, the waves may be moving with divergent characteristics (21).

FIG. 2c is a simplified depiction of the pressure pulse/shock wave generator (shock wave head) having an adjustable or exchangeable housing around the pressure wave path in a low energy divergent position. The adjustable housing or water cushion is moved or expanded much beyond f2 position (20) so that highly divergent wave fronts with low energy density values are leaving the exit window (17) and may be coupled to a patient's body. Thus, an appropriate adjustment can change the energy density of a wave front without changing its characteristic.

This apparatus may, in certain embodiments, be adjusted/modified/or the complete shock wave head or part of it may be exchanged so that the desired and/or optimal acoustic profile such as one having wave fronts with focused, planar, nearly plane, convergent or divergent characteristics can be chosen.

A change of the wave front characteristics may, for example, be achieved by changing the distance of the exit acoustic window relative to the reflector, by changing the reflector geometry, by introducing certain lenses or by removing elements such as lenses that modify the waves produced by a pressure pulse/shock wave generating element. Exemplary pressure pulse/shock wave sources that can, for example, be exchanged for each other to allow an apparatus to generate waves having different wave front characteristics are described in detail below.

In certain embodiments, the change of the distance of the exit acoustic window can be accomplished by a sliding movement. However, in other embodiments of the present invention, in particular, if mechanical complex arrangements, the movement can be an exchange of mechanical elements.

In one embodiment, mechanical elements that are exchanged to achieve a change in wave front characteristics include the primary pressure pulse generating element, the focusing element, the reflecting element, the housing and the membrane. In another embodiment, the mechanical elements further include a closed fluid volume within the housing in which the pressure pulse is formed and transmitted through the exit window.

In one embodiment, the apparatus of the present invention is used in combination therapy. Here, the characteristics of waves emitted by the apparatus are switched from, for example, focused to divergent or from divergent with lower energy density to divergent with higher energy density. Thus, effects of a pressure pulse treatment can be optimized by using waves having different characteristics and/or energy densities, respectively.

While the above described universal toolbox of the present invention provides versatility, the person skilled in the art will appreciate that apparatuses that only produce waves having, for example, nearly plane characteristics, are less mechanically demanding and fulfill the requirements of many users.

As the person skilled in the art will also appreciate that embodiments shown in the drawings are independent of the generation principle and thus are valid for not only electro-hydraulic shock wave generation but also for, but not limited to, PP/SW generation based on electromagnetic, piezoceramic and ballistic principles. The pressure pulse generators may, in certain embodiments, be equipped with a water cushion that houses water which defines the path of pressure pulse waves that is, through which those waves are transmitted. In a preferred embodiment, a patient is coupled via ultrasound gel or oil to the acoustic exit window (17), which can, for example, be an acoustic transparent membrane, a water cushion, a plastic plate or a metal plate.

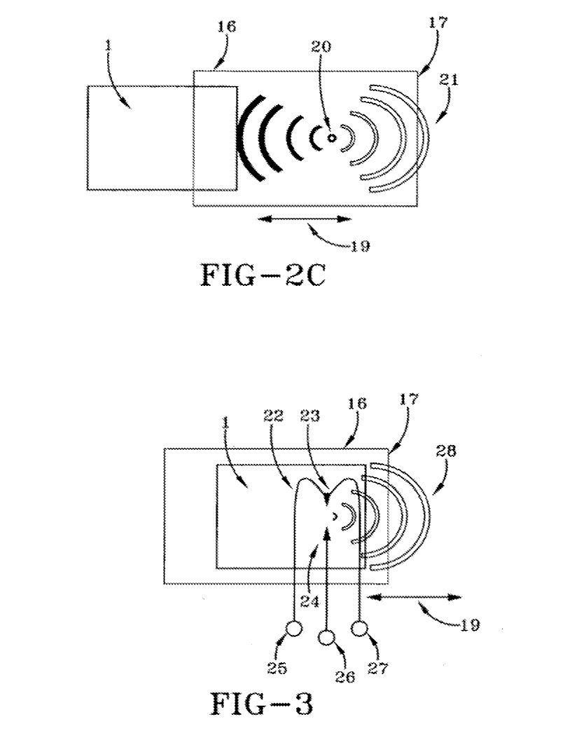

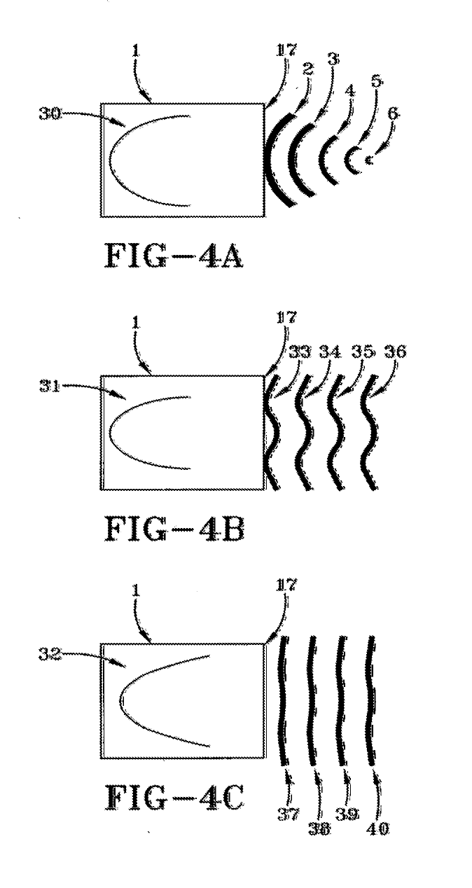

FIG. 3 is a simplified depiction of the pressure pulse/shock wave apparatus having no focusing reflector or other focusing element. The generated waves emanate from the apparatus without coming into contact with any focusing elements. FIG. 3 shows, as an example, an electrode as a pressure pulse generating element producing divergent waves (28) behind the ignition point defined by a spark between the tips of the electrode (23, 24).

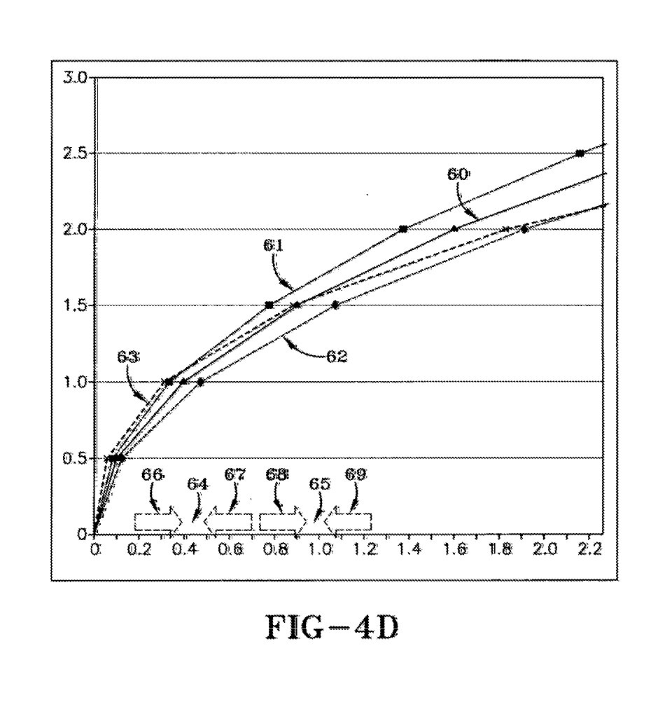

FIG. 4a is a simplified depiction of the pressure pulse/shock wave generator (shock wave head) having as focusing element an ellipsoid (30). Thus, the generated waves are focused at (6).

FIG. 4b is a simplified depiction of the pressure pulse/shock wave generator (shock wave head) having as a focusing element an paraboloid (y2=2px). Thus, the characteristics of the wave fronts generated behind the exit window (33, 34, 35, and 36) are disturbed plane (“parallel”), the disturbance resulting from phenomena ranging from electrode burn down, spark ignition spatial variation to diffraction effects. However, other phenomena might contribute to the disturbance.

FIG. 4c is a simplified depiction of the pressure pulse/shock wave generator (shock wave head) having as a focusing element a generalized paraboloid (yn=2px, with 1.2<n<2.8 and n≠2). Thus, the characteristics of the wave fronts generated behind the exit window (37, 38, 39, and 40) are, compared to the wave fronts generated by a paraboloid (y2=2px), less disturbed, that is, nearly plane (or nearly parallel or nearly even (37, 38, 39, 40)). Thus, conformational adjustments of a regular paraboloid (y2=2px) to produce a generalized paraboloid can compensate for disturbances from, e.g., electrode burn down. Thus, in a generalized paraboloid, the characteristics of the wave front may be nearly plane due to its ability to compensate for phenomena including, but not limited to, burn down of the tips of the electrode and/or for disturbances caused by diffraction at the aperture of the paraboloid. For example, in a regular paraboloid (y2=2px) with p=1.25, introduction of a new electrode may result in p being about 1.05. If an electrode is used that adjusts itself to maintain the distance between the electrode tips (“adjustable electrode”) and assuming that the electrodes burn down is 4 mm (z=4 mm), p will increase to about 1.45. To compensate for this burn down, and here the change of p, and to generate nearly plane wave fronts over the life span of an electrode, a generalized paraboloid having, for example n=1.66 or n=2.5 may be used. An adjustable electrode is, for example, disclosed in U.S. Pat. No. 6,217,531.

FIG. 4d shows sectional views of a number of paraboloids. Numeral 62 indicates a paraboloid of the shape y2=2px with p=0.9 as indicated by numeral 64 at the x axis which specifies the p/2 value (focal point of the paraboloid). Two electrode tips of a new electrode 66 (inner tip) and 67 (outer tip) are also shown in the Figure. If the electrodes are fired and the tips are burning down the position of the tips change, for example, to position 68 and 69 when using an electrode which adjusts its position to compensate for the tip burn down. In order to generate pressure pulse/shock waves having nearly plane characteristics, the paraboloid has to be corrected in its p value. The p value for the burned down electrode is indicate by 65 as p/2=1. This value, which constitutes a slight exaggeration, was chosen to allow for an easier interpretation of the Figure. The corresponding paraboloid has the shape indicated by 61, which is wider than paraboloid 62 because the value of p is increased. An average paraboloid is indicated by numeral 60 in which p=1.25 cm. A generalized paraboloid is indicated by dashed line 63 and constitutes a paraboloid having a shape between paraboloids 61 and 62. This particular generalized paraboloid was generated by choosing a value of n≠2 and a p value of about 1.55 cm. The generalized paraboloid compensates for different p values that result from the electrode burn down and/or adjustment of the electrode tips.

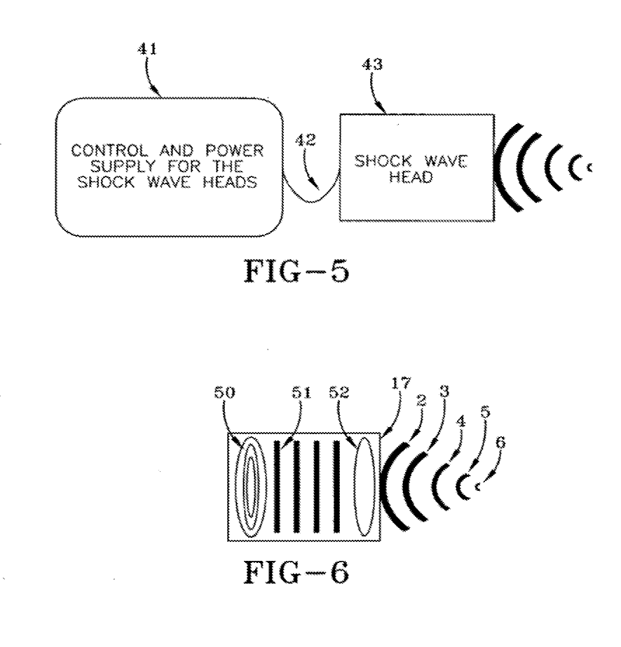

FIG. 5 is a simplified depiction of a set-up of the pressure pulse/shock wave generator (43) (shock wave head) and a control and power supply unit (41) for the shock wave head (43) connected via electrical cables (42) which may also include water hoses that can be used in the context of the present invention. However, as the person skilled in the art will appreciate, other set-ups are possible and within the scope of the present invention.

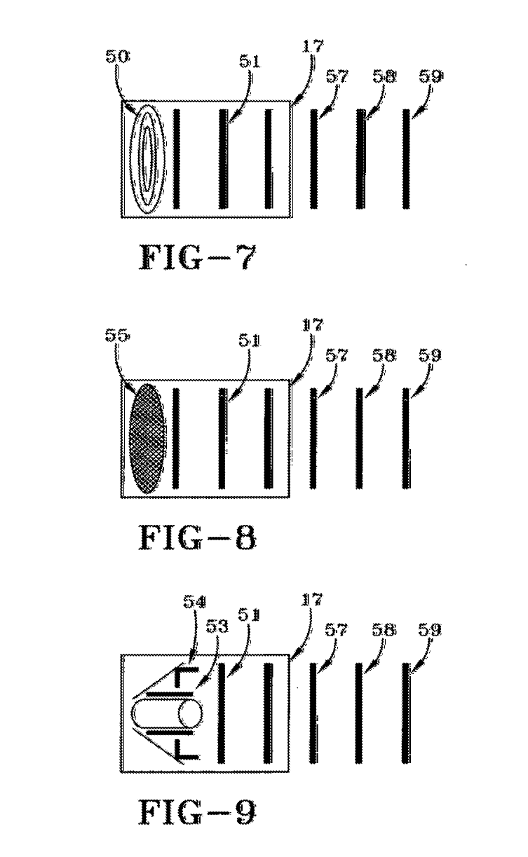

FIG. 6 is a simplified depiction of the pressure pulse/shock wave generator (shock wave head) having an electromagnetic flat coil 50 as the generating element. Because of the plane surface of the accelerated metal membrane of this pressure pulse/shock wave generating element, it emits nearly plane waves which are indicated by lines 51. In shock wave heads, an acoustic lens 52 is generally used to focus these waves. The shape of the lens might vary according to the sound velocity of the material it is made of. At the exit window 17 the focused waves emanate from the housing and converge towards focal point 6.

FIG. 7 is a simplified depiction of the pressure pulse/shock wave generator (shock wave head) having an electromagnetic flat coil 50 as the generating element. Because of the plane surface of the accelerated metal membrane of this generating element, it emits nearly plane waves which are indicated by lines 51. No focusing lens or reflecting lens is used to modify the characteristics of the wave fronts of these waves, thus nearly plane waves having nearly plane characteristics are leaving the housing at exit window 17.

FIG. 8 is a simplified depiction of the pressure pulse/shock wave generator (shock wave head) having an piezoceramic flat surface with piezo crystals 55 as the generating element. Because of the plane surface of this generating element, it emits nearly plane waves which are indicated by lines 51. No focusing lens or reflecting lens is used to modify the characteristics of the wave fronts of these waves, thus nearly plane waves are leaving the housing at exit window 17. Emitting surfaces having other shapes might be used, in particular curved emitting surfaces such as those shown in FIGS. 4a to 4c as well as spherical surfaces. To generate waves having nearly plane or divergent characteristics, additional reflecting elements or lenses might be used. The crystals might, alternatively, be stimulated via an electronic control circuit at different times, so that waves having plane or divergent wave characteristics can be formed even without additional reflecting elements or lenses.

FIG. 9 is a simplified depiction of the pressure pulse/shock wave generator (shock wave head) comprising a cylindrical electromagnet as a generating element 53 and a first reflector having a triangular shape to generate nearly plane waves 54 and 51. Other shapes of the reflector or additional lenses might be used to generate divergent waves as well.

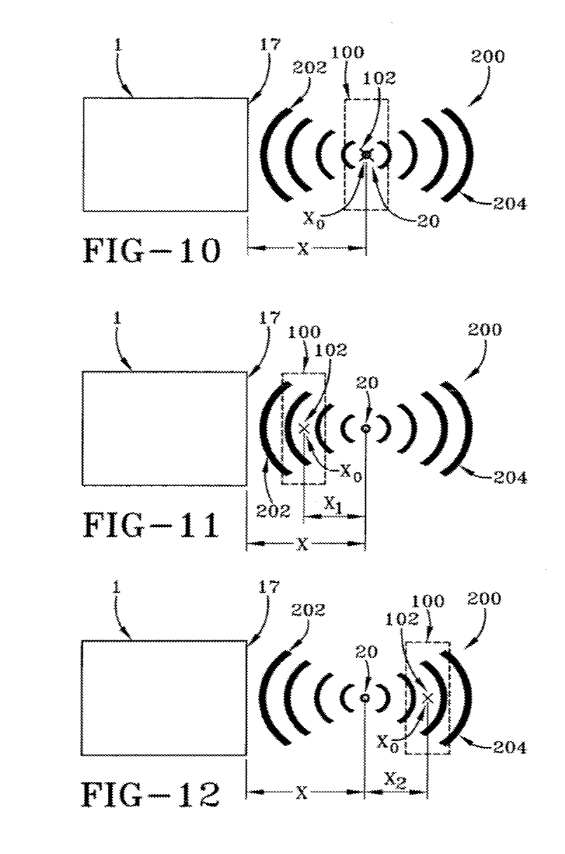

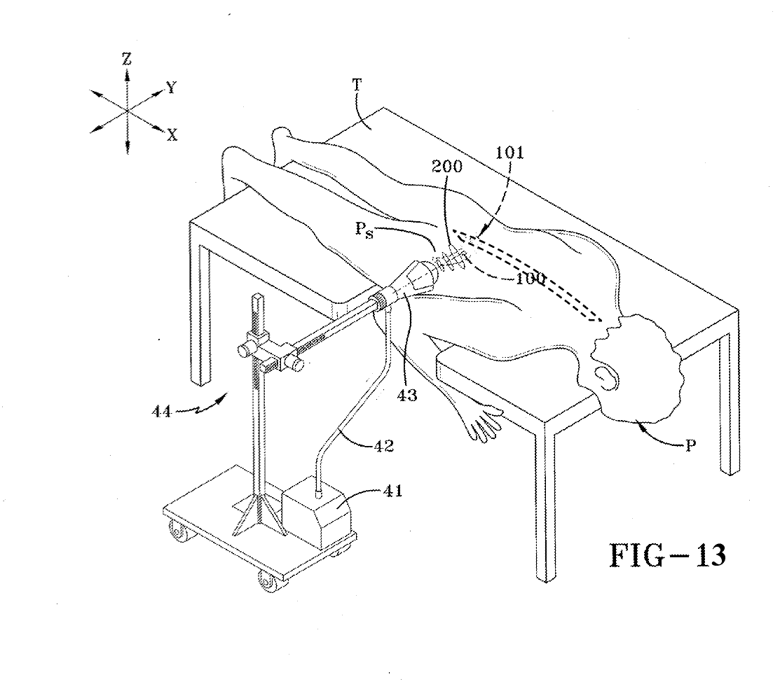

With reference to FIGS. 10, 11 and 12 a schematic view of a shock wave generator or source 1 is shown emitting a shock wave front 200 from an exit window 17. The shock wave front 200 has converging waves 202 extending to a focal point or focal geometric volume 20 at a location spaced a distance X from the generator or source 1. Thereafter the wave front 200 passes from the focal point or geometric volume 20 in a diverging wave pattern as has been discussed in the various other FIGS. 1-9 generally.

With particular reference to FIG. 10 a tissue 100 is shown generally centered on the focal point or volume 20 at a location X₀ within the tissue 100. In this orientation the emitted waves are focused and thus are emitting a high intensity acoustic energy at the location X₀. This location X₀ can be anywhere within or on the organ. Assuming the tissue 100 is a brain tissue having a tumorous mass 102 at location X₀ then the focus is located directly on the mass 102. In one method of treating an infection or mass 102 these focused waves can be directed to destroy or otherwise reduce the mass 102 by weakening the outer barrier shield of the mass 102.

With reference to FIG. 11, the tissue 100 is shifted a distance X toward the generator or source 1. The tissue 100 at location X₀ being positioned a distance X-X₁ from the source 1. This insures the tissue 100 is impinged by converging waves 202 but removed from the focal point 20. When the tissue 100 is tissue this bombardment of converging waves 202 stimulates the cells activating the desired healing response as previously discussed.

With reference to FIG. 12, the tissue 100 is shown shifted or located in the diverging wave portion 204 of the wave front 200. As shown X₀ is now at a distance X₂ from the focal point or geometric volume 20 located at a distance X from the source 1. Accordingly X₀ is located a distance X+X₂ from the source 1. As in FIG. 10 this region of diverging waves 204 can be used to stimulate the tissue 100 which when the tissue is a cellular tissue stimulates the cells to produce the desired healing effect or response.

Heretofore invasive techniques were not used in combination with shock wave therapy primarily because the shock waves were believed to be able to sufficiently pass through interfering body tissue to achieve the desired result in a non-invasive fashion. While this may be true, in many cases if the degenerative process is such that an operation is required then the combination of an operation in conjunction with shock wave therapy only enhances the therapeutic values and the healing process of the patient and the infected organ such that regenerative conditions can be achieved that would include not only revascularization of neurological tissue, but also the heart or other organs wherein sufficient or insufficient blood flow is occurring but also to enhance the improvement of ischemic tissue that may be occupying a portion of the infected tissue or organ. This ischemic tissue can then be minimized by the regenerative process of using shock wave therapy in the fashion described above to permit the tissue to rebuild itself in the region that has been afflicted.

As shown in FIGS. 1-12 the use of these various acoustic shock wave forms can be used separately or in combination to achieve the desired therapeutic effect in treating patients with nerve damage, most importantly to trigger an autonomic nervous system response in a degraded autonomic nervous system caused by a spinal injury or lesion.

Furthermore such acoustic shock wave forms can be used in combination with drugs, chemical treatments, irradiation therapy or even physical therapy and when so combined the stimulated cells will more rapidly assist the body's natural healing response and thus overcomes the otherwise potentially tissue damaging effects of these complimentary procedures.

The present invention provides an apparatus for an effective treatment of indications, which benefit from high or low energy pressure pulse/shock waves having focused or unfocused, nearly plane, convergent or even divergent characteristics. With an unfocused wave having nearly plane, plane, convergent wave characteristic or even divergent wave characteristics, the energy density of the wave may be or may be adjusted to be so low that side effects including pain are very minor or even do not exist at all.

In certain embodiments, the apparatus of the present invention is able to produce waves having energy density values that are below 0.1 mJ/mm² or even as low as 0.000 001 mJ/mm². In a preferred embodiment, those low end values range between 0.1-0.001 mJ/mm². With these low energy densities, side effects are reduced and the dose application is much more uniform. Additionally, the possibility of harming surface tissue is reduced when using an apparatus of the present invention that generates unfocused waves having planar, nearly plane, convergent or divergent characteristics and larger transmission areas compared to apparatuses using a focused shock wave source that need to be moved around to cover the affected area. The apparatus of the present invention also may allow the user to make more precise energy density adjustments than an apparatus generating only focused shock waves, which is generally limited in terms of lowering the energy output. Nevertheless in some cases the first use of a high energy focused shock wave targeting a treatment zone may be the best approach followed by a transmission of lower energy unfocused wave patterns.

The treatment of the above mentioned tissue, organ or body of a patient is believed to be a first time use of acoustic shock wave therapy in the preventive pre-exposure or post-exposure to neurological tissues or organs or nerve damage or degeneration of said tissues or organs for the purpose of activating a degraded autonomic nervous system response.

It will be appreciated that the apparatuses and processes of the present invention can have a variety of embodiments, only a few of which are disclosed herein. It will be apparent to the artisan that other embodiments exist and do not depart from the spirit of the invention. Thus, the described embodiments are illustrative and should not be construed as restrictive.

The use of acoustic shock waves to patients exposed to neurological infections or nerve trauma stimulates a cellular response of the treated tissues as well as a cellular response in the surrounding tissue. This response activates otherwise dormant cells to increase the body's own defense mechanisms, allowing the cells to limit the migration of the infection and resultant tissue damage, but also to initiate the healing process. This feature means that the treating physician has the added benefit of a patient whose body will be strengthened to mitigate damage to otherwise healthy tissues and organs.

This means the physician can use these antibiotic treatments with far less adverse reactions if he combines the treatments with one or more exposures to acoustic shock waves either before introducing chemical antibiotic agents or shortly thereafter or both. This further means that the patient's recovery time should be greatly reduced because the patient treated with shock waves will have initiated a healing response that is much more aggressive than heretofore achieved without the cellular stimulation provided by pressure pulse or shock wave treatments. The current use of medications to stimulate such cellular activity is limited to absorption through the bloodstream via the blood vessels. Acoustic shock waves stimulate all the cells in the region treated activating an almost immediate cellular release of infection fighting and healing agents. Furthermore, as the use of otherwise conflicting chemicals is avoided, adverse side effects can be limited to those medicaments used to destroy the infectious cells. In other words the present invention is far more complimentary to such antibiotic treatments in that the stimulation of otherwise healthy cells will greatly limit the adverse and irreversible effects on the surrounding non-infected tissues and organs.

A further benefit of the use of acoustic shock waves is there are no known adverse indications when combined with the use of other medications or drugs. In fact the activation of the cells exposed to shock wave treatments only enhances cellular absorption of such medication making these drugs faster acting than when compared to non-stimulated cells. As a result, it is envisioned that the use of one or more medicaments prior to, during or after subjecting the patient to acoustic shock waves will be complimentary to the treatment or pre-conditioning treatment for nerve damage. It is further appreciated that certain drug therapies can be altered or modified to lower risk or adverse side effects when combined with a treatment involving acoustic shock waves as described above.

In an experimental acoustic shock wave treatment therapy of a paralyzed patient having a C5-C6 complete spinal cord injury, the injury having occurred approximately seven years prior to treatment. The patient was treated along the spinal cord injury and below with a dosage of 2000 shocks at 0.1 mJ/mm² on at least 12 occasions with healing intervals of 1 week between treatments. This resulted in an initial re-activation of the autonomic nervous system first evidenced by the functional ability to perspire occurring after the fifth treatment, then at week 10 the patient's diaphragm regained 100 percent functionality from a pre-treatment severely comprised condition. Between weeks 5 and 10, the patient regained muscle control in the back and abdomen enabling the patient to rotate or twist his back and perform exercises such as stomach crunches. He can move his upper limbs since week 5. Currently, he can leg press up to 80 pounds. The treatment is continuing for an estimated six more months. Due to muscle atrophy, the fine motor skills of the limbs will continue to improve with exercise, something this patient could not do in the past. Additional goals of this study are to treat that lower portion of the spinal cord to reactivate nerve control of the bladder and bowels. This will enable this patient to hopefully regain normal functioning to allow removal of catherized bag for urination.

The current activation of the autonomic nervous system while not completely re-established is approaching progressively normal use. The patient now feels the sensation of pain in all extremities. The regaining of limb functionality to pre-accident 100 percent efficiency has decreased his risk of pneumonia while allowing him to take deep full breaths. This increased oxygenation of the red blood cells. All these physical functionalities were achieved by the response to the autonomic nervous system activation. Additional clinical trials are currently being conducted on five other patients. All five are showing similar early improvements.

Variations in the present invention are possible in light of the description of it provided herein. While certain representative embodiments and details have been shown for the purpose of illustrating the subject invention, it will be apparent to those skilled in this art that various changes and modifications can be made therein without departing from the scope of the subject invention. It is, therefore, to be understood that changes can be made in the particular embodiments described which will be within the full intended scope of the invention as defined by the following appended claims.

What is claimed is:

1. A method of treating a patient having a nerve injury or spinal cord injury or spinal cord lesions, or disease or any pathology that has compromised an autonomic nervous system of a patient to stimulate regeneration or reactivation of the autonomic nervous system by activating or regenerating a degraded autonomic nervous system comprises the steps of:
treating the patient with injured or damaged nerves;
activating an acoustic shock wave generator or source to emit acoustic shock waves from a shock wave head; and
administering an effective exposure of acoustic shock waves in a pulse or wave pattern having an energy density less than 1.0 mJ/mm² per shock wave directly onto a treatment zone in a region extending from the medulla oblongata in the lower brain stem to the lower end of the spinal cord, the administered shock waves activating an autonomic nervous system response in either a parasympathetic nervous system or a sympathetic nervous system or both systems of the autonomic nervous system.

2. The method of treating a patient having a nerve injury or spinal cord injury or spinal cord lesions, or disease or any pathology that has compromised an autonomic nervous system of a patient to stimulate regeneration or reactivation of the autonomic nervous system by activating or regenerating a degraded autonomic nervous system of claim 1 wherein the autonomic nervous system response included an improvement in one or more visceral functions.

3. The method of treating a patient having a nerve injury or spinal cord injury or spinal cord lesions, or disease or any pathology that has compromised an autonomic nervous system of a patient to stimulate regeneration or reactivation of the autonomic nervous system by activating or regenerating a degraded autonomic nervous system of claim 2 wherein the improvement in one or more visceral functions includes one or more of cardio-vascular performance, digestion, respiratory performance, salivation, perspiration, pupillary dilation, micturition and sexual arousal.

4. The method of treating a patient having a nerve injury or spinal cord injury or spinal cord lesions, or disease or any pathology that has compromised an autonomic nervous system of a patient to stimulate regeneration or reactivation of the autonomic nervous system by activating or regenerating a degraded autonomic nervous system of claim 1 wherein the administered acoustic shock waves cause an increase nitric oxide in the treatment zone.

5. The method of treating a patient having a nerve injury or spinal cord injury or spinal cord lesions, or disease or any pathology that has compromised an autonomic nervous system of a patient to stimulate regeneration or reactivation of the autonomic nervous system by activating or regenerating a degraded autonomic nervous system of claim 1 wherein the administered acoustic shock waves cause a chemical release of neurotransmitters from the nerves.

6. The method of treating a patient having a nerve injury or spinal cord injury or spinal cord lesions, or disease or any pathology that has compromised an autonomic nervous system of a patient to stimulate regeneration or reactivation of the autonomic nervous system by activating or regenerating a degraded autonomic nervous system of claim 5 wherein the neurotransmitters released are epinephrine or acetylcholine or nitric oxide.

7. The method of treating a patient having a nerve injury or spinal cord injury or spinal cord lesions, or disease or any pathology that has compromised an autonomic nervous system of a patient to stimulate regeneration or reactivation of the autonomic nervous system by activating or regenerating a degraded autonomic nervous system of claim 1 wherein the patient has a spinal injury resulting in complete or partial paralysis or a disease or other pathology that has impaired the autonomic nervous system or spinal cord or nerve function.

8. The method of treating a patient having a nerve injury or spinal cord injury or spinal cord lesions, or disease or any pathology that has compromised an autonomic nervous system of a patient to stimulate regeneration or reactivation of the autonomic nervous system by activating or regenerating a degraded autonomic nervous system of claim 7 wherein the patient has a reduced pulmonary function or has lost the ability to perspire in any portion of the body, typically below the site of a spinal injury or disease.

9. The method of treating a patient having a nerve injury or spinal cord injury or spinal cord lesions, or disease or any pathology that has compromised an autonomic nervous system of a patient to stimulate regeneration or reactivation of the autonomic nervous system by activating or regenerating a degraded autonomic nervous system of claim 8 wherein the improvement is evidenced by activation of the diaphragm muscles.

10. The method of treating a patient having a nerve injury or spinal cord injury or spinal cord lesions, or disease or any pathology that has compromised an autonomic nervous system of a patient to stimulate regeneration or reactivation of the autonomic nervous system by activating or regenerating a degraded autonomic nervous system of claim 9 wherein the improvement is evidenced by increased or near normal lung function and improved breathing.

11. The method of treating a patient having a nerve injury or spinal cord injury or spinal cord lesions, or disease or any pathology that has compromised an autonomic nervous system of a patient to stimulate regeneration or reactivation of the autonomic nervous system by activating or regenerating a degraded autonomic nervous system of claim 7 wherein the improvement is evidenced by active perspiration.

12. The method of treating a patient having a nerve injury or spinal cord injury or spinal cord lesions, or disease or any pathology that has compromised an autonomic nervous system of a patient to stimulate regeneration or reactivation of the autonomic nervous system by activating or regenerating a degraded autonomic nervous system of claim 7 wherein the improvement is evidenced by limb movement.

13. The method of treating a patient having a nerve injury or spinal cord injury or spinal cord lesions, or disease or any pathology that has compromised an autonomic nervous system of a patient to stimulate regeneration or reactivation of the autonomic nervous system by activating or regenerating a degraded autonomic nervous system of claim 7 wherein the improvement is evidenced by normal bladder control and urination.

14. The method of treating a patient having a nerve injury or spinal cord injury or spinal cord lesions, or disease or any pathology that has compromised an autonomic nervous system of a patient to stimulate regeneration or reactivation of the autonomic nervous system by activating or regenerating a degraded autonomic nervous system of claim 1 further comprises the step of causing stem cells to migrate and differentiate to the treatment zone.
